(19) United States
(12) Patent Application Publication
(10) Pub. No.: US 2008/0161405 A1
(43) Pub. Date: Jul. 3, 2008

(54) Biguanide Composition and Method of Treatment and Prevention of Infections

(76) Inventors: Erning Xia, Penfield, NY (US); Hongna Wang, Fairport, NY (US); Praveen Tyle, Pittsford, NY (US)

Correspondence Address: Bausch & Lomb Incorporated, One Bausch & Lomb Place, Rochester, NY, 14604-2701, US

(21) Appl. No.: 11/962,146

(22) Filed: Dec. 21, 2007

Related U.S. Application Data
(60) Provisional application No. 60/882,620, filed on Dec. 29, 2006.

(51) Int. Cl. A61K 31/155; A61P 31/00

(52) U.S. Cl. 514/635

(57) ABSTRACT

The present invention includes a package for a medicament for treating infectious disease. The package comprises a first vessel and a second vessel. The first vessel contains a solid mixture of a biguanide antimicrobial agent and a polyol. The polyol has a melting point above room temperature. The ratio of biguanide antimicrobial agent to polyol is a maximum of about 1:5 and a minimum of about 1:1000. The solid mixture comprises less than 10 wt. % water based upon its total weight. The second vessel comprises an aqueous diluent. The present invention includes methods of manufacturing the package and methods of treating infection.

### CROSS-REFERENCE

This application claims the benefit of Provisional Patent Application No. 60/882,620 filed Dec. 29, 2006, which is incorporated by reference herein.

### BACKGROUND OF THE INVENTION

1. Field of the Invention

This invention relates to the stabilization of formulations to treat ocular and other topical infections.

2. Discussion of the Related Art

Ocular infections are conditions that require treatment. Depending on the structures which are involved and the infecting organism, ocular infections may range from discomfort (conjunctivitis) to serious pain and vision loss (keratitis). Ocular infections include bacterial, viral, fungal and amoebal species.

A clinically effective antimicrobial agent is one that is potent against a particular microbe yet is not toxic to human tissue.

Such an agent that is toxic against a wide range of microbes, yet has relatively little toxicity against human tissue is considerably more valuable.

Biguanide antimicrobial agents have been used to preserve ophthalmic solutions and demonstrate relatively low toxicity in ocular tissues. Biguanide antimicrobial agents include polyhexamethylene biguanide, chlorhexidine and Alexidine.

To effectively preserve an ophthalmic composition, sufficient preservative is necessary to prevent growth of S. aureus, P. aeruginosa and E. coli bacteria and C. albicans and A. niger fungi over the shelf life of the product. Typically, a clinically effective formulation will contain the lowest amount of a preservative required to accomplish the desired effect. Between 0.5 ppm and 3.0 ppm of a biguanide has been used to preserve most ophthalmic solutions.

Biguanide antimicrobial agents have been used as disinfectant solutions for contact lenses. To be considered a disinfectant, a solution needs sufficient antimicrobial agent to kill S. aureus, P. aeruginosa and S. marcescens bacteria and C. albicans and F. solani fungi over the shelf life of the product. Furthermore, the solution must show efficacy in disinfecting contact lenses using the disinfecting regimen that is recommended on the product. This regimen is arrived at through data which supports the disinfecting properties described above.

Disinfecting solutions containing antimicrobial agents include ReNu® Multiplus sold by Bausch & Lomb, Rochester, N.Y. ReNu® Multiplus is a multipurpose cleaning, conditioning and disinfecting solution for contact lenses that contains 1 ppm of polyhexamethylene biguanide.

Disinfecting solutions such as the one mentioned above are ophthalmically safe solutions. They are safe to administer to the eye of a patient. Contact lenses that have been rinsed with these solutions are placed in the eye. However, these solutions are not approved for use as a medicament in the eye. There is no evidence to suggest that the level of antimicrobial agent in a multipurpose contact lens solution would be effective to treat ocular infection.

Several studies have been conducted on the effectiveness of polyhexamethylene biguanide and/or chlorhexidine for treatment of Acanthamoebal keratitis and Fungal keratitis.

In Schuster, et al., “Opportunistic Amoebae: Challenges In Prophylaxis And Treatment,” Drug Resistance Updates: Reviews And Commentaries In Antimicrobial And Anticancer Chemotherapy, Vol. 7, No. 1, 41-51 (February 2004), Acanthamoeba keratitis, a non-opportunistic infection of the cornea, was found to respond to treatment with chlorhexidine gluconate and polyhexamethylene biguanide, in combination with propamidine isothionate (Brolene), hexamidine (Desomodine), or neomycin.

In Rama, et al., “Bilateral Acanthamoeba keratitis with late recurrence of the infection in a corneal graft: a case report,” European Journal of Ophthalmology, Vol. 13, No. 3, 311-14 (April 2003), recurrences of Acanthamoeba keratitis in both eyes were successfully treated with a combination of hexamidine and neomycin, and with polyhexamethylene biguanide, respectively.

Panda, et al., “Role of 0.02% polyhexamethylene biguanide and 1% povidone iodine in experimental Aspergillus keratitis,” Cornea, Vol. 22, No. 2, 138-41, (March 2003) showed that polyhexamethylene biguanide (0.02%) is a moderately effective drug for experimental Aspergillus keratitis.

Fiscella, et al. “Polyhexamethylene Biguanide (PHMB) in the Treatment of Experimental Fusarium Keratomycosis,” Cornea, Vol. 16, No. 4, 447-49 (1997) teaches that a 0.02% solution of PHMB was effective at reducing fungal growth in a rabbit model.

Sharma, et al., “Patient characteristics, diagnosis and treatment of non-contact lens related Acanthamoeba keratitis,” British Journal of Ophthalmology, Vol. 84, No. 10, 1103-1108 (2000) illustrates the combination of polyhexamethylene biguanide and chlorhexidine. See also Alexandrakis, et al., “Amebic Keratitis Due to Vahlkampfia Infection Following Corneal Trauma,” Arch. Ophthalmology, Vol. 116, 950-51 (July 1998); Dua, et al., “Non-Acanthamoeba Amebic Keratitis,” Cornea, Vol. 17, No. 6, 675-77 (1998); Bobo, et al., “Les Keratites Amibiennes,” Med. Trop., Vol. 55, No. 4bis, 439-43 (1995) (English Abstract); Burger et al., “Acantamoeben-keratitis: Een erstige ooginfectie in optomst,” Pharamceutisch Weekblad, Vol. 131, No. 3, 72-77 (1996) (English Abstract); Prajna et al., Effect of Topical 2% Polyhexamethylene Biguanide on Nocardial Keratitis Associated with Scleritis,” Indian Journal of Ophthalmology, Vol. 46, No. 4, 251-52 (December, 1998); Messick, et al., “In-vitro activity of polyhexamethylene biguanide (PHMB) against fungal isolates associated with infective keratitis,” J. Antimicrob. Chemother., Vol. 44, 297-98 (1999).

Alexidine in addition to polyhexamethylene biguanide and chlorhexidine was shown to have activity against acanthamoeba keratitis at a minimum inhibitory concentration of 6.3 μg/ml for cysts and trophazoites. Pyott, et al., “Acanthamoeba keratitis: first recorded case from a Palestinian patient with trachoma,” British Journal of Ophthalmology, Vol. 80, 849 (1996). In Connor, et al., “Guanidines, Diamidines and Biguanides: Towards a Rational Therapy for Acanthamoeba Keratitis,” J. Pharm. Pharmacol., Vol. 47, No. 12, 1007 (1995) indicates that biguanides, including chlorhexidine, alexidine and poly hexamethylene biguanide were considered favorable for first line treatment of Acanthamoeba Keratitis. Chlorhexidine was considered the most preferred. See also, Seal, “Acanthamoeba keratitis update—incidence, molecular epidemiology and new drugs for treatment,” Eye, Vol. 17, 893-905 (2003).

WO 97/00076 discloses a composition that contains poly(hexamethylene biguanide salts. The formulations include buffers such as borate/boric acid, phosphate buffer, and acetate/citrate buffer. The pH disclosed in this reference is 7.4.

WO2005/097094 discloses a composition for topical administration (i.e. administration to the skin of a human or animal) of an antimicrobial agent in a formulation with a chelating agent and a buffer. The pH of the solution is disclosed between 3 and 9, but preferably between 5 and 8. Specific formulations have a pH no lower than 6.7 and are primarily alkaline.

Shelf life is an important issue for pharmaceuticals that treat ocular infection. Particularly, no less than 90% of an active agent can deteriorate over a two-year period of time to be approved by the Food and Drug Administration. Biguanides are somewhat unstable in aqueous solutions and degrade over time.

Consequently, there is a need for a dosage form that will stabilize an ophthalmic solution for a long period of time, preferably without chemical stabilizers. The present invention addresses this and other needs.

### SUMMARY OF THE INVENTION

The present invention is a package for a topical medicament for treating infectious disease. The package comprises a first vessel and a second vessel. The first vessel contains a solid mixture of a biguanide antimicrobial agent and a polyol. The polyol has a melting point above 0° C. The ratio of biguanide antimicrobial agent to polyol is a maximum of about 1:5 and a minimum of about 1:1000. The first vessel contains less than about 10 wt. % water based upon the total weight of the solid mixture. The second vessel comprises an aqueous diluent.

In another embodiment, there is a method of making a medicament for topically treating infectious disease. The method comprises providing a first vessel containing a solid mixture of a biguanide antimicrobial agent and polyol having a melting point above 0° C. The ratio of biguanide antimicrobial agent to polyol is a maximum of about 1:5 and a minimum of about 1:1000. The solid mixture in the first vessel has less than about 10 wt. % water based upon the total weight of the biguanide antimicrobial agent. The method includes an additional step of providing a second vessel containing an aqueous diluent.

In still another embodiment, there is a method of treating an ocular infection. The method comprises applying a composition to an affected area of a subject, which composition comprises a biguanide antimicrobial agent, a polyol, and a diluent; wherein the polyol has a melting point above 0° C.; the polyol and the biguanide antimicrobial agent are contained in a first vessel; a ratio of biguanide antimicrobial agent to polyol is in a range from about 1:5 to about 1:1000; the diluent is contained in a second vessel; and contents of the first and second vessels are combined to form the composition shortly before the composition is applied to said affected area. In certain embodiment, the contents of the first vessel are a solid mixture. The contents of the first vessel comprise less than about 10 wt. % water based upon the total weight of the solid mixture. In one aspect, the composition is administered topically to treat an infectious condition, for example, an ocular infection.

In yet another embodiment, the first vessel is made of glass, polypropylene, polyethylene or poly(ethylene teraphthalate). Optionally, the second vessel is made of glass, polypropylene, polyethylene or poly(ethylene teraphthalate). Preferably, the first vessel is enclosed in the second vessel. Typically, the first vessel is breakable.

In one embodiment, the solid mixture is capable of dissolving in the mixture upon shaking for a maximum time of about 60 seconds, about 40 seconds, about 30 seconds, about 20 seconds or about 10 seconds.

In another embodiment, the polyol is a C₃-C₂₀ polyol. The polyol is preferably selected from the group consisting of sucrose, glucose, xylitol, mannitol or sorbitol.

In one embodiment, the ratio of diluent to polyol is a maximum of about 1:5 and a minimum or about 1:1000. Typically, the ratio of diluent to polyol is a maximum of about 1:5, about 1:10, about 1:20, about 1:50 and/or a minimum of about 1:1000, about 1:500, about 1:200, about 1:100, about 1:50.

In another embodiment, the solid mixture and/or diluent comprise a buffer—preferably, a borate buffer.

The liquid mixture, typically, comprises an osmolality adjusting agent such that the combination of the solid mixture and the diluent will result in an osmolality that is a minimum of about 240 mOsm/Kg and a maximum of about 400 mOsm/Kg.

In still another embodiment, the solid mixture and/or diluent comprises a surfactant, a viscosity enhancing agent a penetration enhancer and/or a buffer system.

In yet another embodiment, the diluent comprises less than a stabilizing amount of ethylenediaminetetraacetic acid or hydroxyalkylphosphonate—preferably no ethylenediaminetetraacetic acid or hydroxyalkylphosphonate. In another embodiment, the combined solution contains less than a stabilizing amount of ethylenediaminetetraacetic acid or hydroxyalkylphosphonate—preferably no ethylenediaminetetraacetic acid or hydroxyalkylphosphonate.

In another embodiment, the biguanide antimicrobial agent is selected from the group consisting of poly(hexamethylene biguanide), alexidine and chlorhexidine. Preferably the biguanide antimicrobial agent is alexidine. Optionally, the amount of alexidine is a minimum of about 0.1 ppm and a maximum of about 5000 ppm based upon the total weight of the solid mixture and the diluent.

### BRIEF DESCRIPTION OF THE DRAWING

FIG. 1 shows the loss of Alexidine from solutions having various initial concentrations of Alexidine, stored in glass containers.

### DETAILED DESCRIPTION OF THE INVENTION

Biguanide antimicrobial agents, and particularly Alexidine, are somewhat unstable in aqueous solutions. The present invention provides longer shelf-life of the medicament containing alexidine. In one embodiment, the desired stability is obtained without chemical stabilizers.

Alexidine is a biguanide antimicrobial agent that is defined by the formula 1,1′-hexamethylene-bis[5-(2-ethylhexyl)biguanide]. By biguanide antimicrobial agent it is meant an antimicrobial agent that has biguanide substituents and has antimicrobial properties in an ophthalmically safe amount. Suitable biguanide antimicrobial agents include but are not limited to 1,1′-hexamethylene-bis[5-(p-chlorophenyl)biguanide] (Chlorhexidine) or water soluble salts thereof, 1,1′-hexamethylene-bis[5-(2-ethylhexyl)biguanide] (Alexidine) or water-soluble salts thereof, and poly(hexamethylene biguanide) (PHMB).

In one embodiment, the amount of biguanide antimicrobial agent in the topical composition is a maximum of about 1 ppm and a minimum of about 0.1 wt. %. Typically, the amount of antimicrobial agent in the multipurpose solution is a minimum of about 4.5 ppm, about 5 ppm, about 10 ppm, about 15 ppm or about 20 ppm. Typically, the amount of antimicrobial agent in the ophthalmic solution is a maximum of about 1000 ppm, about 500 ppm, about 300 ppm, about 100 ppm, about 75 ppm or about 50 ppm.

In one embodiment, the amount of antimicrobial agent is sufficient to provide an in eye concentration that is a minimum of about 0.001 ppm and a maximum of about 100 ppm. Preferably, the in-eye concentration is a minimum of about 0.1 ppm, about 1 ppm, about 5 ppm, about 10 ppm or about 50 ppm and is a maximum of about 90 ppm, about 80 ppm, about 70 ppm or about 60 ppm. The in-eye concentration is determined by sampling a portion of the lachrymal fluid after 3 blinks of the eye. The in-eye concentration is based upon the concentration of antimicrobial agent. It may also be significantly affected by the concentration of viscosity adjusting agents, penetration enhancers, surfactants, if any, and other agents that may cause the antimicrobial agent to remain in solution or bind to the corneal tissue of the eye. In one embodiment, the in-eye concentration of antimicrobial agent is 10% of the concentration of antimicrobial agent in the storage solution.

The present invention is a package that contains a first vessel and a second vessel. The first vessel contains a solid mixture of a biguanide antimicrobial agent and a polyol. The polyol has a melting point above 0° C. The ratio of biguanide antimicrobial agent to polyol is a maximum of about 1:5 and a minimum of about 1:1000. The first vessel contains less than 10 wt. % water based upon the total weight of the solid mixture. The second vessel comprises an aqueous diluent. The packaging separates the solid mixture from the aqueous diluent until some time as it is desired to solubilize the solid mixture with the aqueous diluent. Preferably, the package can remain for two years or longer without loss of more than 10% Alexidine in the solid mixture. After the aqueous diluent and the solid mixture are combined, they form a combined solution. The combined solution can be used for a treatment period, i.e. a minimum of about four days, about one week, about ten days or about two weeks and a maximum of about two months, about one month, about two weeks, about ten days, about one week.

In one embodiment, the solid mixture is lyophilized by preparing a liquid mixture with the solid components and lyophilizing the liquid mixture until only the components that are solid at the desired storage temperature remain. Lyophilization techniques are well known in the art.

The solid mixture additionally contains a polyol in one embodiment. The polyol provides needed bulk to improve the handling of the biguanide and/or assists with rapid dissolution of the biguanide antimicrobial agent. The polyol of the present invention is typically a polyol containing 3 to 20 carbon atoms. Preferably, the polyol contains 6-18 carbon atoms. The polyol of one embodiment is selected from the group consisting of poly(ethylene glycol), propylene glycol, sorbitol, manitol, xylitol and monosaccharides, disaccharides, oligosaccharides and neutral polysaccharides. In one preferred embodiment, the polyol is selected from the group consisting of sorbitol, mannitol, xylitol, dextrose, trehalose, cyclodextrin, mannose and sucrose. The polyol preferably has a melting point above room temperature so that the solid mixture remains in solid form during storage. Preferably, the polyol has a melting point that is above about 30° C., about 35° C., about 40° C. and about 45° C. The ratio of biguanide antimicrobial agent to polyol is a minimum of about 1:5 and a maximum of about 1:1000. Typically, the ratio of diluent to polyol is a maximum of about 1:5, about 1:10, about 1:20, about 1:50 and/or a minimum of about 1:1000, about 1:500, about 1:200, about 1:100, about 1:50.

The solid mixture preferably contains little or no water during storage in the first vessel. Thus, the first vessel typically contains less than 5 wt. % water based upon the total weight of the solid mixture. Preferably the solid mixture contains less than 1 wt. % water, 0.5 wt. % water; 0.01 wt. % water based upon the total weight of the solid mixture.

In one embodiment, the solid mixture and/or diluent contain a buffer or buffer combination. One or more conventional buffers can alternatively be employed to obtain the desired pH value. Suitable buffers include, but not limited to, citrate buffer, histidine, phosphate buffer, maleate buffer, cacodylate buffer, bis-tris buffer, carbonate buffer, imidazole buffer, ADA buffer, ACES buffer, PIPES buffer, MOPSO buffer, HEPES buffer, MOPS buffer, BES buffer, TES buffer, MOBS buffer, DIPSO buffer, TAPSO buffer, triethanolamine buffer, pyrophosphate buffer, HEPPSO buffer, POPSO buffer, tricine buffer, hydrazine buffer, glycylglycine buffer, Trizma buffer, HEPBS buffer, TAPS buffer, TABS buffer, AMPSO buffer, taurine buffer and borate buffer. Generally, buffers will be used in amounts ranging from about 0.05 to about 2.5 weight percent, and preferably, from about 0.1 to about 1.5 weight percent. Preferably, the buffer will have a pKa that is from about 6.0 to about 9. Preferably, the buffer will have a pKa that is a minimum of 6.5 or 7 or 7.5 and/or a maximum of about 9, about 8.5 or about 8. Optionally, a proportional amount of buffer can be present in the diluent.

Additionally, other components such as tonicity adjusting agents, viscosifiers, stabilizers, humectants, penetration enhancers and stabilizers can be found in either the solid mixture and/or the diluent. If the ingredients are present in the solid mixture, they should have a melting point above the recommended storage temperature and are readily dissolvable In one embodiment the desired storage temperature is 0° C. or 4° C. In another embodiment, the storage temperature is room temperature. In one embodiment, the polyol has a desired melting point above room temperature, such as above about 30 C, above about 35° C., above about 40° C., or above about 45° C.

The solid mixture and/or diluent optionally include a penetration enhancer. The penetration enhancer generally acts to make the cell membranes less rigid and therefore more amenable to allowing passage of drug molecules between cells. The penetration enhancers preferably exert their penetration enhancing effect immediately upon application to the eye and maintain this effect for a period of approximately five to ten minutes. The penetration enhancers and any metabolites thereof must also be non-toxic to ophthalmic tissues. One or more penetration enhancers will generally be utilized in a minimum amount of about 0.01 weight percent and/or a maximum of about 10 wt. % based upon the total volume of the combined solution after the solid mixture is combined with the diluent.

The preferred penetration enhancers are saccharide surfactants, such as dodecylmaltoside (“DDM”), and monoacyl phosphoglycerides, such as lysophosphatidylcholine. The saccharide surfactants and monoacyl phosphoglycerides, which may be utilized, as penetration enhancers in the present invention are known compounds. The use of such compounds to enhance the penetration of ophthalmic drugs is described in U.S. Pat. No. 5,221,696 the entire contents of which are incorporated by reference into the present specification. Other penetration enhancers that are ophthalmically acceptable include ethylenediaminetetraacetic acid and other Ca2+ chelating agents, cytochalasin B (a group of small molecules bind specifically to actin microfilaments), cyclodextrin and derivatives.

The present invention, in one embodiment, provides a stable formulation without stabilizing chemicals (stabilizing enhancers). In this embodiment, the effective shelf life of the antimicrobial agent is extended in the solid mixture by a minimum of about 10 percent of the shelf life without the stabilizer compared to a solution that comprises a fully hydrated formulation without a chemical stabilizer. In another embodiment, the antimicrobial agent is extended by a minimum of about 20 percent, about 40 percent, about 80 percent, about 100 percent or about 200 percent.

The present invention enhances the stability of the composition for long-term storage and in one embodiment requires no stabilizer. However, a stabilizer can be added to further boost the stability after the solid mixture and the diluent have been combined into a combined solution. The present invention, when used in the presence of a stabilizer will further extend the shelf-life of the combined solution.

A stabilizer is a compound that prevents the chemical degradation of an active agent in solution. Examples of stabilizers that are effective in an aqueous solution include but are not limited to ophthalmically acceptable antioxidants (ascorbate, methionine, citric acid, BHT), complexing agents (cyclodextrin and derivatives, hyaluronic acid, citric acid), non-ionic surfactants (poloxamines such as Tetronics® 908, tyloxapol) and chelating agents and salts thereof (hydroxyl alkyl phosphonate, sodium edentate).

In another embodiment, the preferred stabilizer is present in a minimum amount of about 0.001 wt. %, about 0.005 wt. %, about 0.01 wt. % and/or a maximum amount of about 0.5 wt. %, about 0.3 wt. %, about 0.1 wt. %, about 0.08 wt. %, about 0.05 wt. %, about 0.03 wt. %, about 0.01 wt. %.

In another embodiment, the solid mixture and/or diluent of the present invention contains a delivery vehicle that increases the mean residence time of the active agent in the eye and/or enhances penetration in the eye. U.S. Pat. Nos. 6,884,788 or 6,261,547 or 5,800,807 or 5,618,800 or 5,496,811 disclose various ophthalmic delivery vehicles the teachings in these patents are incorporated by reference in their entirety.

The viscosifiers are optionally used in the present invention to increase the mean residence time of the active ingredient in the eye. Viscosifiers can be present in the solid mixture and/or the diluent. With the aid of a viscosifier, liquid drops can be used having a viscosity that is a minimum of about 2 cps and a maximum of about 100 cps. Viscosifiers can be used to formulate liquid gels that have a viscosity ranging from about 100 cps to about 1000 cps. Ophthalmic gels will generally have a viscosity in excess of 1000 cps. Regardless, the viscosifier is utilized to ensure an adequate mean residence time in the eye. Any synthetic or natural polymer, which is capable of forming a viscous or a solid insert, may be utilized. In addition to having the physical properties required to form a viscous gel or solid insert, the polymers must also be compatible with tissues of the eye. The polymers must also be chemically and physically compatible with the above-described active agent and other components of the compositions.

Polymers, which satisfy the foregoing criteria, are referred to herein as “ophthalmically acceptable viscous polymers.” Examples of suitable polymers include: natural polysaccharides and gums, such as alginate, carrageenan, guar, karaya, locust bean, tragacanth agarose and xanthan; modified naturally occurring polymers such as carboxymethylcellulose, hydroxyethylcellulose, hydroxypropylcellulose, hydroxypropylmethylcellulose, methylcellulose, hydroxypropylmethylguar and carboxymethyguar, synthetic polymers, such as carboxy vinyl polymers, polyvinyl alcohol and polyvinyl pyrrolidone.

In addition, proteins and synthetic polypeptides that form viscous gels and are ophthalmically acceptable can be used to provide better bioavailability. Typically, proteins that can be used include: gelatin, collagen, albumin, whey protein and casein.

Polymers which have high molecular weights and, most importantly, physical properties that mimic the physical properties of the mucous secretions found in the eye are referred to herein as being “mucomimetic.” A preferred class of mucomimetic polymers is carboxy vinyl polymers having molecular weights in the range of from about 50,000 to about 6,000,000. The polymers have carboxylic acid functional groups and preferably contain between 2 and 7 carbon atoms per functional group. The gels that form during preparation of the ophthalmic polymer dispersion have a viscosity between about 1,000 to about 300,000 centipoise (cps). Suitable carboxy vinyl polymers include those called carbomers, e.g., Carbopol[R] (B. F. Goodrich Co., Cleveland, Ohio). Specifically preferred are carbomer 934, 940, 970, 974 and 980. Such polymers will typically be employed in an amount between about 0.05 and about 8.0 wt %, depending on the desired viscosity of the composition.

The diluent and/or solid mixture optionally or alternatively includes a tonicity adjusting agent to approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride, dextrose, mannose, glycerin, calcium and magnesium chloride. These agents are typically used individually in amounts that are a minimum of about 0.01 wt. % or about 0.2 wt. % and/or a maximum of about 2.5 wt. % or 1.5 wt. %. Preferably, the tonicity agent is employed in an amount to provide a final osmotic value that is a minimum of 200 mOsm/kg, 220 mOsm/kg and/or a maximum of about 450 mOsm/kg, 350 mOsm/kg or about 320 mOsm/kg.

The solid mixture and/or the diluent can optionally include a humectant. The humectant that is present in the solid mixture preferably has a melting point greater than the recommended storage temperature. The humectant is present to provide moisture to the eye. A first class of humectants is polymer humectants. Examples of suitable humectants include for example but are not limited to poly(vinyl alcohol) (PVA), poly(N-vinylpyrrolidone) (PVP), cellulose derivatives and poly(ethylene glycol). As disclosed in U.S. Pat. No. 6,274,133, cationic cellulosic polymers include for example but are not limited to water soluble polymers commercially available under the CTFA (Cosmetic, Toiletry, and Fragrance Association) designation Polyquaternium-10, including the cationic cellulosic polymers available under the trade name UCARE® Polymers from Amerchol Corp., Edison, N.J., such as for example but not limited to Polymer JR™. Generally, these cationic cellulose polymers contain quaternized N,N-dimethylamino groups along the cellulosic polymer chain.

Another suitable class of humectants is non-polymeric humectants. Examples may include glycerin, propylene glycol, and other non-polymeric diols and glycols. The specific quantities of humectants used in the invention will vary depending upon the application. However, the humectants will typically be included in an amount from about 0.01 to about 5 weight percent, preferably from about 0.1 to about 2 weight percent.

It will be understood that some constituents possess more than one functional attribute. For example, cellulose derivatives are suitable polymeric humectants, but are also referred to as “viscosity increasing agents” to increase viscosity of the composition if desired. Glycerin is a suitable non-polymeric humectant but it also may contribute to adjusting tonicity.

The second vessel contains an aqueous diluent. The aqueous diluent contains the amount of water that is required to dissolve the solid mixture. The amount of water is measured to result in the desired concentration of the biguanide antimicrobial agent for the resulting solution. In one embodiment, the aqueous diluent is purified water. In another embodiment, the aqueous diluent is a physiological saline solution. In still another embodiment, the diluent is a buffered saline solution. Non-limiting examples of buffered saline solution include phosphate buffered saline or borate buffered saline.

In still another embodiment, one or more of all of the solid ingredients, except the biguanide antimicrobial agent and at least a portion of the polyol, is optionally present in the diluent. For example, some or all of the buffer or buffering system, viscosity enhancing agent, surfactant, stabilizer, penetration enhancer, tonicity adjusting agent, and demulcent are optionally present in the diluent.

In one embodiment, the package comprises a first vessel containing a solid mixture and a second vessel containing a diluent. The term vessel means a closed container that is liquid tight unless the vessel is ruptured or intentionally opened. The first vessel and the second vessel are attached. “Attached” means that the two vessels accompany one another in a single package unit. In one embodiment, the first vessel is contained within the second vessel. Preferably the first vessel is breakable.

Upon mixing the solid mixture and the diluent, the solid mixture should be readily dissolvable. In one embodiment, the mixture is dissolvable in the diluent upon agitating for a maximum of about 60 seconds, about 40 seconds, about 30 seconds, about 20 seconds or about 10 seconds. The solid mixture is combined with the diluent to form a composition shortly (such as no more than about 30 minutes, or 15 minutes, or 5 minutes) before it is to be applied to an affected area of a patient in need of treatment or control of infection (such as ocular infection).

It will be understood that the present invention is typically applied by administering a solution to the eye of a patient in the form of eye drops, liquid gels or viscous gels. In one embodiment, one to four drops are applied to each eye. Preferably two drops are applied to each eye. In one embodiment, the drops are placed directly on the eye. In another embodiment, the drops are placed in the conjuntival sac beneath the eye.

Typically, the drops are administered a minimum of once daily, two times daily, three times daily, or four times daily and a maximum of once hourly, once every two hours, once every three hours, once every four hours, once every six hours.

### EXAMPLES

### Example 1

### Alexidine Lyophilized Formulations

The following formulation listed in Table 1 was prepared in a separate vessel for lyophilized powder and diluent.

TABLE 1
Lyophilized Formula 1

| Ingredients | w/w(%) |
|---|---|
| Lyophilized Powder | |
| Alexidine 2HCl | 5 |
| Mannitol | 80 |
| Boric Acid | 15 |
| Diluent | |
| CMC (MV) | 1 |
| Pluronic ® F127 | 0.2 |
| EDTA | 0.1 |
| BAK (benzakonium chloride) | 0.01 |
| Borate buffer (0.05 M, pH 7.4) | 0.09 |
| Purified Water | q.s. 100 wt. % |

Optionally, all of the solid components can be added to the solid mixture that is lyophilized. The diluent in this embodiment is preferably purified water. This embodiment is preferable when the diluent is to be supplied by the user and is not provided with the solid mixture. However, such an embodiment would rely upon the user to dispense the appropriate amount of fluid for required concentrations of the combined solution. It is preferable in one embodiment, to provide the diluent in the exact amount to be used to avoid the user from having to measure and dispense the correct amount of water.

The lyophilized powder and the diluent are combined. The lyophilized powder rapidly dissolves in the diluent. The solutions are rehydrated at day 3, 5 and 13. The amount of Alexidine is analyzed in solution. The amount of Alexidine in each of the test solutions showed less Alexidine was lost than would be expected if Alexidine was stored for the same amount of time in an aqueous solution in a glass container.

### Example 2

### Alexidine Concentration Changes in Glass Vessels Over Time

Alexidine has been known to decrease in concentration in solutions that are stored in glass containers. It has been proposed that the Alexidine non-specifically binds to the glass surface of the container. An experiment was conducted to determine the extent and rate that Alexidine disappears from solution in glass containers, as exhibited by a decrease in UV/VIS radiation absorbance by Alexidine.

Five concentrations of Alexidine solution were prepared in Pyrex class A volumetric flasks and identified as Alexidine Solutions 1-5.

TABLE 2
Alexidine Concentration Over Time

| Solution | Concentration | |
|---|---|---|
| Alexidine Solution 1 | 3.125 | ppm |
| Alexidine Solution 2 | 6.25 | ppm |
| Alexidine Solution 3 | 12.5 | ppm |
| Alexidine Solution 4 | 25 | ppm |
| Alexidine Solution 5 | 50 | ppm |
| Control Solution | 0 | |

The solutions were made in a borate buffer solution consisting of 0.85% boric acid, 0.22% sodium chloride, and 0.09% sodium borate (pH=7.31, osmolality=213 mOsm/kg). A control solution was prepared with boric acid, sodium chloride and sodium borate.

Alexidine solution was scanned from 200-400 nm on a Shimadzu UV-160 UV-Visible Spectrophotometer to determine maximum adsorption for Alexidine. It was found to occur at 236 nm. The UV/VIS was background corrected using zero concentration standard (control solution). A standard curve (@ 236 nm) was generated by freshly prepared Alexidine Solutions 1-5.

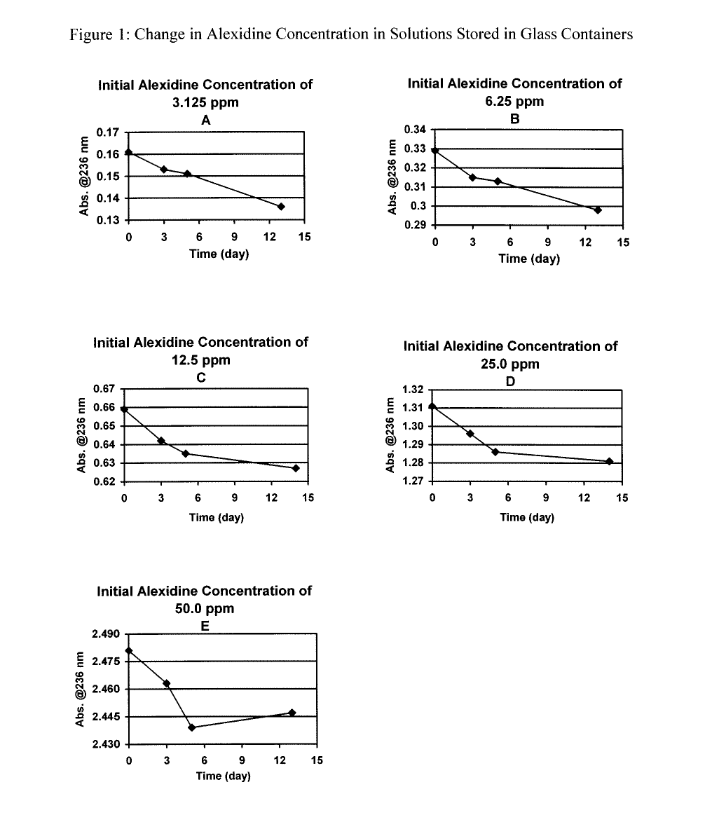

The Alexidine Solutions 1-5 were then kept at room temperature in individual flasks, and analyzed at day 0, 3, 5 and 13. The Alexidine concentration decreased over time for each Alexidine Solution 1-5, as exhibited by the decrease in absorbance, as shown in FIG. 1.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A package for a medicament for treating infectious disease comprising:
a first vessel containing a solid mixture of a biguanide antimicrobial agent and a polyol having a melting point above 0° C., wherein the ratio of biguanide antimicrobial agent to polyol is a maximum of about 1:5 and a minimum of about 1:1000, the first vessel contains less than 10 wt. % water based upon the total weight of the solid mixture; and
a second vessel containing an aqueous diluent.

2. The package of claim 1, wherein the first vessel is enclosed in the second vessel.

3. The package of claim 1, wherein the first vessel is breakable.

4. The package of claim 1, wherein the solid mixture is capable of being dissolved in the diluent upon agitating for a maximum time of about 60 seconds.

5. The package of claim 1, wherein the first vessel and the second vessel are attached.

6. The package of claim 1, wherein the polyol is a C3-C20 polyol.

7. The package of claim 1, wherein the polyol is selected from the group consisting of mannose, dextrose, sucrose, xylitol, mannitol, trehalose, cyclodextrin or sorbitol.

8. The package of claim 1, wherein the ratio of diluent to polyol is a maximum of about 1:10 and a minimum or about 1:500.

9. The package of claim 1, wherein the solid mixture comprises a buffer.

10. The package of claim 1, wherein the solid mixture comprises a borate buffer.

11. The package of claim 1, wherein the liquid mixture comprises an osmolality adjusting agent such that the combination of the solid mixture and the diluent will result in an osmolality that is a minimum of 240 mOsm/Kg and a maximum of 400 mOsm/Kg.

12. The package of claim 1, wherein the diluent comprises a viscosity enhancing agent.

13. The package of claim 1, wherein the diluent comprises a penetration enhancer.

14. The package of claim 1, wherein the diluent comprises less than a stabilizing amount of ethylenediaminetetraacetic acid or hydroxyalkylphosphonate.

15. The package of claim 1, wherein the diluent comprises a buffer.

16. The package of claim 1, wherein the biguanide antimicrobial agent is selected from the group consisting of poly(hexamethylene biguanide), alexidine and chlorhexidine.

17. The package of claim 1, wherein the biguanide antimicrobial agent is alexidine.

18. The package of claim 20, wherein the amount of alexidine is a minimum of about 0.1 ppm and a maximum of about 5000 ppm based upon the total weight of the solid mixture and the diluent.

19. A method of making a medicament for treating infection disease comprising:
(a) providing a first vessel containing a solid mixture of a biguanide antimicrobial agent and polyol having a melting point above 0° C., wherein the ratio of biguanide antimicrobial agent to polyol is a maximum of about 1:5 and a minimum of about 1:1000, wherein the solid mixture in the first vessel comprises less than 10 wt. % water based upon the weight of the solid mixture;
(b) providing a second vessel containing an aqueous diluent; and
(c) combining the solid mixture and the diluent to form the medicament shortly before use.

20. A method of treating a topical infection comprising:
applying to an affected area of a subject a composition that comprises a biguanide antimicrobial agent, a polyol, and a diluent; wherein the polyol has a melting point above 0° C.; the biguanide antimicrobial agent and polyol are contained in a first vessel as a solid mixture; a ratio of biguanide antimicrobial agent to polyol is in a range from about 1:5 to about 1:1000; the solid mixture comprises less than 10 wt % water based on the weight of the solid mixture; the diluent is contained in a separate second vessel; and contents of the first and second vessels are combined to form the composition shortly before the composition is applied to said affected area.
